(19) United States
(12) Patent Application Publication
(10) Pub. No.: US 2004/0152746 A1
(43) Pub. Date: Aug. 5, 2004

(54) Treatment of scarring and related conditions using ppar-gamma activators

(76) Inventors: Hazel Judith Bardsley, Konstanz (DE); Stephen Bentley, Cambridge (GB); David Cavalla, Cambridge (GB)

Correspondence Address: Saliwanchik Lloyd & Saliwanchik, Suite A 1, 2421 N W 41st Street, Gainesville, FL, 32606-6669, US

(21) Appl. No.: 10/473,920

(22) PCT Filed: Apr. 30, 2002
(86) PCT No.: PCT/GB02/01976

(30) Foreign Application Priority Data
Apr. 30, 2001 (GB) 0110584.0
Nov. 26, 2001 (GB) 0128300.1

(51) Int. Cl. A61K 31/427; A61K 31/557

(52) U.S. Cl. 514/369; 514/573; 514/374

(57) ABSTRACT

An activator of PPARγ (such as pioglitazone) is useful for the treatment of a condition affecting the skin characterised by disordered fibroblast or myofibroblast function, excessive matrix production, modular fasciitis or Dupuytren's Contracture. The invention is particularly useful for treating keloids and hypertrophic scarring.

### FIELD OF THE INVENTION

This invention relates to a new use for compounds effective as agonists at the peroxisome proliferator activator receptor gamma (PPARγ).

### BACKGROUND OF THE INVENTION

Excess scar formation after trauma or surgery can lead to body disfigurement, prurititis and pain and organ dysfunction. Hypertrophic scars and keloids are skin fibrotic conditions that can occur even after minor injury such as ear piercing. They are unique to humans and are characterised by excessive deposition of collagen in the dermis and subcutaneous tissue. Keloids range in size from papules a few millimetres in diameter to football size or larger. Differences between keloids, hypertrophic scars and normal scars include appearance, histologic morphology and cellular function in response to growth factors.

Hypertrophic scars are characterised by raised fibrous connective tissue in the dermis and adjacent subcutaneous tissue after traumatic or burn healing. Keloids typically comprise a nodular, frequently lobulated firm movable, non-encapsulated mass of peculiar hyperplastic fibrous connective tissue consisting of-densely collagenous material found in the dermis and adjacent subcutaneous tissue after skin injury. Clinically, keloids are defined as scars growing beyond the normal confines of original wounds which, unlike hypertrophic scars, rarely regress with time. It has been estimated that approx 15-20% of Blacks, Hispanics and Orientals suffer from keloids and work is underway to understand a possible genetic predisposition.

Dermal fibroblasts and myofibroblasts play a major role in scar formation. In this specification, unless further qualified, the term fibroblast is taken to cover fibroblasts and/or myofibroblasts or any other subtype of fibroblast.

Keloid fibroblasts produce high levels of collagen, fibronectin, elastin and proteoglycan. Collagen synthesis in keloids is 20 times greater than in normal unscarred skin. It is known that keloid-derived fibroblasts exhibit as much as a four-fold increase in the rate of fibronectin biosynthesis. Keloid fibroblasts also show aberrant responses, compared with normal fibroblasts to metabolic modulators such as glucocorticoids, hydrocortisone, growth factors and phorbol esters. The altered response of keloid fibroblasts, to these metabolic modulators is thought to contribute to the pathogenesis of keloid formation. Fibroblasts from hypertrophic scars also display a moderate elevation in collagen production in vitro, however their response to the metabolic modulators is similar to normal fibroblasts. There is evidence that fibroblasts from hypertrophic scars might represent a hyperproliferactive phenotype resulting from multiple stimulatory effects present in the wound environment. This phenotype can be reversed once the overstimulation, such as overabundance of growth factors, is lifted. Keloid fibroblasts however represent a unique phenotype that is switched on irreversibly after wounding. There is also evidence that there is an inbalance in lipid biology in keloids.

After wounding, healing mechanisms are the same regardless of the cause of the damage and may be considered in three phases; an immediate haemostatic phase, an early granulation and re-epithelial phase, and a later phase of dermal repair and remodelling. The haemostatic processes involve formation of a platelet plug and fibrin clot. The early granulation and re-epithelial phase takes place up to 21 days after injury depending on the wound site and size. Platelet-derived growth factors stimulate fibroblasts to produce granulation tissue, comprising a collagen matrix well supplied with capillary vessels and growth of epidermal cells leading to re-epithelialisation of the wound surface. The collagen matrix undergoes strengthening during the dermal repair and remodelling phase and there is a reduction in vascularity. This phase can continue for up to 2 years after injury (Martindale, 32^{nd} Edition). Granulation tissue formation involves replication and migration of fibroblasts from normal tissues to the area of inflammation and the modulation of at least a proportion of them towards the myofibroblastic phenotype. Angiogenesis (supply of capillary vessels) takes place in a co-ordinated way and granulation tissue acquires its typical features. An important part of wound healing is wound contraction or closure. The myofibroblast (or granulation tissue fibroblast) is responsible for the generation of contractile force associated with wound contraction and is characterised by the presence of alpha-smooth muscle actin-containing stress fibres. When the wound closes, a gradual evolution towards scar tissue takes place which involves disappearance of vascular cells and myofibroblasts. This phenomenon ends with the establishment of a scar.

When granulation tissue cells are not eliminated, there is development of pathological scarring, i.e. hypertrophic and keloid scars which are both characterised by a high degree of cellularity. It has been shown that the reduction in cell number observed during the transition between granulation tissue and scar is achieved to a great extent by apoptosis. A progressive apoptotic wave is responsible for the gradual disappearance of granulation tissue myofibroblasts. It appears that apoptosis of granulation tissue cells takes place after wound closure, and affects target cells consecutively rather than producing a simple wave of cell disappearance (Gabbiani, Pathol. Res. Pract. 192(7): 708-711, 1996). Recently it was shown that hypertrophic scars have greater numbers of fibroblasts staining for alpha smooth muscle actin (implicating them as myofibroblasts) than normal skin or healed donor sites. Furthermore over time as the hypertrophic scar remodels, the number of fibroblasts and myofibroblasts reduces (Nedelec et al, Surgery 130(5): 798-808, 2001).

In fibroblast cultures derived from keloids, the percentage of apoptotic cells was shown to be 1% for all cell lines. These levels were 50% lower than those found in cell cultures containing normal fibroblasts. Also fibroblasts derived from the centre of keloids proliferate faster than those from the periphery. This suggests that keloids could be caused by an abnormal balance between cell growth and cell death (Luo et al, Surgery 107: 87-96, 2001). Other work has shown that fibroblasts from keloids have lower rates of apoptosis and that there was evidence of mutation in the gene p53, an important tumour suppressor gene linked to apoptotic pathways (Ladin et al, Wound Repair Regen. 6(1): 28-37, 1998).

There is now mounting evidence that pathological scarring states could be caused by persistent presence of a population of myofibroblasts. In addition, there is evidence that, in scleroderma, an unpleasant skin condition, the dermal myofibroblast predominates, compared with normal skin. Scieroderma is an uncommon fibrotic disorder with a high morbidity. It is an aggressive disorder, in which there is excessive immune activation resulting in extensive skin fibrosis, due to extensive fibroblast proliferation and endothelial cell dysfunction. It often develops from Raynaud's syndrome.

Treatment strategies including surgery can be effective for hypertrophic scars; however the treatment of keloids is poor. Surgery is not a good option as recurrence rates without adjuvant therapy such as steroids vary from 45% to 100%. Treatment of keloids with intralesional steroids such as triamcinolone is often ineffective and the majority of patients have recurrences within a year. There are also side-effects. There does not appear to be any way of giving prophylaxis in keloid-prone patients. Nor is there a way of preventing hypertrophic scars at present. It is clear that new treatments and prophylactic measures are required for abnormal scarring, particularly keloids.

The PPARγ receptor is a subtype of the PPAR (peroxisome proliferator activated receptor) family of nuclear hormone receptors. It has been shown to function as important regulator in lipid and glucose metabolism, adipocyte differentiation, inflammatory response and energy homeostasis.

The thiazolidinediones rosiglitazone and pioglitazone are used for the treatment of insulin resistance in type II diabetes. Thiazolinedione activators of PPARγ have also been shown to have anti-proliferative and anti-inflammatory effects in vascular myocytes and macrophages. Furthermore, troglitazone has been shown to have anti-proliferative effects on keratinocytes in psoriasis. In this disease keratinocyte hyperproliferation and immune dysfunction are major components. Such compounds and their utility in therapy are described in U.S. Pat. No. 5,594,015, U.S. Pat. No. 5,824,694, U.S. Pat. No. 5,925,657 and U.S. Pat. No. 5,981,586.

Conversely, activators of the alpha subtype of the PPAR (PPARα), which include such compounds as clofibrate and gemfibrozil, have been described in U.S. Pat. No. 6,060,515 for their ability to enhance epithelial barrier development. Acting through an effect on trans-epithelial water loss, hypertrophic scars and keloids are among a large number of examples of skin conditions that are claimed to be susceptible to such treatment.

### SUMMARY OF THE INVENTION

Surprisingly, it has been found that a PPARγ activator such as pioglitazone has the ability to reduce myofibroblast cell numbers. It may therefore be used to treat keloid and hypertrophic scar formation and treat scleroderma. This is based on the understanding that selected apoptosis of myofibroblasts or a inhibitory effect on fibroblast differentiation to myofibroblasts could prevent the formation of, or accelerate the remodelling of hypertrophic scars towards a normal scar, or could facilitate tissue remodelling of a keloid so that it reduces in size or disappears, and treat the symptoms of scleroderma which include skin oedema, skin thickening and tightening followed by atrophy and contractures.

By means of this invention, keloids, hypertrophic scars, scleroderma, other skin diseases associated with abnormal myofibroblast function or nodular fasciitis, or Dupuytren's Contracture can be treated, e.g. controlled or prevented.

### BRIEF DESCRIPTION OF THE DRAWINGS

FIG. 1: A: 0 μM, B: 0.1 μM, C: 1 μM, D: 10 μM Pioglitazone in fibroblast differentiation medium supplemented with 1 ng/ml TGF-beta1. Arrows indicate examples of DAB-positive myofibroblasts.

FIG. 2: A: Fibroblasts cultured in differentiation medium supplemented with 1 ng/ml TGF-beta1 in the absence of pioglitazone (x100), B: Differentiated fibroblasts cultured with pioglitazone (10 uM) (x100), C: High magnification (x200) differentiated fibroblasts indicating cells which are thought to be myofibroblasts based on their abundant and enlarged cytoskeletal filaments (arrows), D: High magnification (x200) of pioglitazone treated (10 uM) differentiated fibroblasts.

FIG. 3: A: Myofibroblast from culture not exposed to pioglitazone treatment showing the typical large, spread morphology and prominent myofibrils (arrowhead). B: Pioglitazone-treated culture showing an α-smooth muscle positive cell (large arrow) which has clearly shrunk and rounded up compared to the surrounding normal fibroblasts. There is also evidence of membrane blebbing (arrow heads) which is suggestive of apoptosis rather than necrotic death. C and D: Pioglitazone-treated cultures. Myofibroblasts labelled with a large arrow show strong yet diffuse cytoplasmic staining.

### DESCRIPTION OF PREFERRED EMBODIMENTS

Any PPARγ activator may be used in this invention provided it has the desired activity. Well known activators of this receptor include the thiazolidinediones, troglitazone, pioglitazone, rosiglitazone and ciglitazone. Other non-thiazolidinedione compounds have recently been identified such as the phenyl alkanoic acids described in WO97/31907 and WO00/08002, the oxazoles and thiazoles described in WO99/58510, the oximinoalkanoic acids described in WO01/38325, benzoic acid derivatives described in WO01/12612, the sulphonamides described in WO99/38845, the β-aryl-α-oxysubstituted alkylcarboxylic acids described in WO00/50414 and the quinolines described in WO00/64876 and WO00/64888. In addition, the natural compound 15-deoxy-Δ-12,14-prostaglandin J2 has also been found to be a ligand for PPARγ and to have effects mediated through this receptor (Forman et al., Cell 93(5): 813-819, 1995). Similar effects have also been found for metabolites of 15-deoxy-Δ-12,14-prostaglandin J2 (Kliewer et al., Cell 83(5): 813-819,1995) and for various fatty acids and eicosanoids (Kliewer et al, PNAS USA 94(a): 43184323, 1997).

Despite the structural variation tolerated by PPARγ, there is a substantial similarity in biological effect due to activation of this receptor. PPAR agonists share a common binding mode to their receptors. Despite differences in the chemical structure of these agonists, the acidic headgroups of these agonist ligands accept a hydrogen bond from a tyrosine residue in the AF2 helix and/or a histidine or tyrosine residue in helix-5 (see description in WO01/17994). Compounds with the ability to activate PPARγ receptors can be expected to be useful in this invention. Preferred agents for this use in the invention include pioglitazone, rosiglitazone, ciglitazone, troglitazone, isaglitazone, darglitazone and englitazone. It will be understood that a prodrug or metabolite or such a compound can be used.

Formulations and Administration

For use in the invention, therapeutic compounds are typically administered to human patients topically, by intralesional or subcutaneous injection. Oral and parenteral administration are used in appropriate circumstances apparent to the practitioner. Preferably, the compositions are administered in unit dosage forms suitable for single administration of precise dosage amounts.

To prepare a topical formulation, a therapeutically effective concentration of the compound is placed in a dermatological vehicle as is known in the art. The amount of the therapeutic compound to be administered and the compound's concentration in the topical formulations depend upon the vehicle selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

The concentration of the therapeutic compound for topical formulation is in the range of about 0.01 mg/ml to about 100 mg/ml. Typically, the concentration of the therapeutic compound for topical formulation is in the range of about 0.1 mg/ml to about 10 mg/ml. Solid dispersions of the therapeutic compound as well as solubilized preparations can be used. Thus, the precise concentration is subject to modest experimental manipulation in order to optimize the therapeutic response. About 1,000 mg of therapeutic compound per 100 grams of vehicle is useful in the treatment of skin lesions to provide a 1.0% weight/weight (w/w) formulation. Suitable vehicles include oil-in-water or water-in-oil emulsions using mineral oils, petrolatum and the like as well as gels such as hydrogel.

Alternative topical formulations include shampoo preparations, oral paste, and mouth wash preparations. Concentrations of therapeutic compound are typically as stated above for topical formulations.

The therapeutic compound is optionally administered topically by the use of a transdermal therapeutic system (see Barry, Darmatological Formulations, Marcel Dekker, 1983, p. 181 and literature cited therein). While such topical delivery systems have been designed largely for transdermal administration of low molecular weight drugs, by definition they are capable of percutaneous delivery. They may be readily adapted to administration of the therapeutic compounds of the invention by appropriate selection of the rate-controlling microporous membrane.

Ordinarily, an aqueous aerosol is made by formulating an aqueous solution or suspension of the therapeutic compound together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

For oral administration, either solid or fluid unit dosage forms can be prepared. For preparing solid compositions such as tablets, the therapeutic compound is mixed into a formulation with conventional ingredients such as talc, magnesium stearate, dicalcium phosphate, magnesium aluminum silicate, calcium sulfate, starch, lactose, acacia, methylcellulose, and functionally similar materials pharmaceutical diluents or carriers. Capsules are prepared by mixing the therapeutic compound with an inert pharmaceutical diluent and filling the mixture into a hard gelatin capsule of appropriate size. Soft gelatin capsules are prepared by machine encapsulation of a slurry of the therapeutic compound with an acceptable vegetable oil, light liquid petrolatum or other inert oil.

Fluid unit dosage forms for oral administration such as syrups, elixirs and suspensions can be prepared. The water-soluble forms can be dissolved in an aqueous vehicle together with sugar, aromatic flavoring agents and preservatives to form a syrup. An elixir is prepared by using a hydroalcoholic (e.g., ethanol) vehicle with suitable sweeteners such as sugar and saccharin, together with an aromatic flavouring agent. Suspensions can be prepared with an aqueous vehicle with the aid of a suspending agent such as acacia, tragacanth, methylcellulose and the like.

Appropriate formulations for parenteral use are apparent to the practitioner of ordinary skill. Usually, the therapeutic compound is prepared in an aqueous solution (discussed below) in a concentration of from about 0.01 to about 100 mg/ml. More typically, the concentration is from about 0.1 to about 10 mg/ml. The formulation, which is sterile, is suitable for various parenteral routes including intra-dermal, intra-articular, intra-muscular, intravascular, and subcutaneous.

In addition to the therapeutic compound, the compositions may include, depending on the formulation desired, pharmaceutically-acceptable, non-toxic carriers or diluents, which include vehicles commonly used to form pharmaceutical compositions for animal or human administration. The diluent is selected so as not to unduly affect the biological activity of the combination. Examples of such diluents which are especially useful for injectable formulations are water, the various saline solutions, Ringer's solution, dextrose solution, and Hank's solution. In addition, the pharmaceutical composition or formulation may include additives such as other carriers; adjuvants; or nontoxic, nontherapeutic, nonimmunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g., Tris or phosphate buffers. Effective amounts of diluents, additives and excipients are those amounts which are effective to obtain a pharmaceutically acceptable formulation in terms of solubility, biological activity, etc.

The term “unit dosage form” refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved and (b) the limitations inherent in the art of compounding such an active material for use in humans and animals.

Examples of unit dosage forms are tablets, capsules, pills, powder packets, wafers, suppositories, granules, cachets, teaspoonsful, tablespoonsful, droppersful, ampoules, vials, aerosols with metered discharges, segregated multiples of any of the foregoing, and other forms as herein described.

Thus, a composition of the invention includes a therapeutic compound which may be formulated with conventional, pharmaceutically acceptable, vehicles for topical, oral or parenteral administration. Formulations may also include small amounts of adjuvants such as buffers and preservatives to maintain isotonicity, physiological and pH stability. Means of preparation, formulation and administration are known to those of skill. See generally Remington's Pharmaceutical Science 15th ed., Mack Publishing Co., Easton, Pa. (1980).

Slow or extended-release delivery systems, including any of a number of biopolymers (biological-based systems), systems employing liposomes, and polymeric delivery systems, can be utilized with the compositions described herein to provide a continuous or long-term source of therapeutic compound. Such slow release systems are applicable to formulations for topical, ophthalmic, oral, and parenteral use.

Therapeutic agents of the invention are usually delivered or administered topically or by transdermal patches for treating hypertrophic or keloidal scarring. Alternatively, oral administration is used. Additionally, the agents can be delivered parenterally, or by direct injection of skin lesions. Parenteral therapy is typically intra-dermal or intravenous.

A preferred way to practice the invention is to apply the therapeutic compound, in a cream or oil based carrier, directly to the scar or lesion. Typically, the concentration of therapeutic compound in a cream or oil is 0.1-10%. Alternatively, an aerosol can be used topically. These compounds can also be orally administered. The thiazolidinedione compound piogitazone is an example of a thiazolidinedione that can be used in this fashion.

In general, the route of administration is topical (including administration to the eye, scalp, and mucous membranes), oral, or parenteral. Topical administration is preferred in treatment of skin lesions, including lesions of the scalp, lesions of the cornea, and lesions of mucous membranes where such direct application is practical. Mouthwash and oral paste formulations can be advantageous for mucous membrane lesions, such as oral lesions and leukoplakia. Oral administration is a preferred alternative for treatment of skin lesions and other lesions discussed above where direct topical application is not as practical, and it is a preferred route for other applications.

Intra-dermal injection is a preferred alternative in the case of treating one or only a few (such as 2-6) lesions. Usually, the compound is delivered in an aqueous solution of about 0.1-10 mg/ml.

An effective quantity of therapeutic compound is employed in treatment. The dosage of compounds used in accordance with the invention varies depending on the compound and the condition being treated. The age, weight, and clinical condition of the recipient patient; and the experience and judgement of the clinician or practitioner administering the therapy are among the factors affecting the selected dosage. Other factors include the route of administration, the patient, the patient's medical history, the severity of the disease process, and the potency of the particular compound. The dose should be sufficient to ameliorate symptoms or signs of the disease treated without producing unacceptable toxicity to the patient.

Broadly, a dosing schedule is from about 0.1 to about 600 mg twice a day. More typically, a single dose is about 1-200 mg of compound given twice a day. A convenient oral dose for an adult patient is 10 mg twice a day. A dosage range for topical treatment is about 0.1% to about 10% (weight/volume) in a cream, gel or oil, applied twice a day. A typical dosage for intra-dermal administration is about 0.1-10 mg per injection per site.

Typically, the dosage is administered at least once a day until a therapeutic result is achieved. Preferably, the dosage is administered twice a day, but more or less frequent dosing can be recommended by the clinician. Once a therapeutic result is achieved, the drug can be tapered or discontinued. Occasionally, side-effects warrant discontinuation of therapy. In general, an effective amount of the compound is that which provides either subjective relief of symptoms or an objectively identifiable improvement as noted by the clinician or other qualified observer.

The foregoing is offered primarily for purposes of illustration. It will be readily apparent to those of ordinary skill in the art that the operating conditions, materials, procedural steps and other parameters of the system described herein may be further modified or substituted in various ways without departing from the spirit and scope of the invention. For example, the invention has been described with human patients as the usual recipient, but veterinary use is also contemplated. Thus, the invention is not limited by the preceding description, but rather by the appended claims. All cited literature is incorporated by reference.

### EXAMPLE 1

Myofibroblast Number After Treatment With Pioglitazone

Human dermal fibroblasts were cultured on sterile cover-slips and allowed to adhere for 24 hours in fibroblast medium, i.e. (1.0 ml L-Glutamine (200 mM; Gibco), 1.0 ml non-essential amino acids, NEAA(100×; Gibco), 1.0 ml penicillin/streptomycin (100×Gibco), 10 ml new-born calf serum, NBCS (Gibco), 87 ml Dulbecco's modified Eagle's Medium, DMEM (Gibco)). At confluence, the medium was changed to myofibroblast differentiation medium, i.e. 1.0 ml L-Glutamine (200 mM; Gibco), 1.0 ml non-essential amino acids, NEAA(100×; Gibco), 10 ml penicillin/streptomycin (100×Gibco), 1 ml ITS (Insulin, Transferrin, Selenium)(100×Gibco)) supplemented with 1 ng/ml TGF-beta1 (Sigma). Myofibroblast differentiation was allowed to proceed for 3 days either in the presence of variable concentrations of pioglitazone or without.

Myofibroblast number was determined by immunohistochemistry according to the following protocol. Cells were washed in phosphate buffered saline (PBS), then fixed in 3% paraformaldehyde for 5 mins followed by quenching in 50 mM ammonium chloride. The cells were then washed and permeabilised with 0.2% Triton X-100 in PBS. All antibodies were diluted into 0.2% fish skin gelatin (FSG) (Sigma). Prior to antibody detection non-specific binding was blocked using 0.2% FSG in PBS. Mouse anti-human alpha-smooth muscle actin (Sigma) was diluted 1:100 and incubated with the cells for 45 mins followed by washing. Rabbit anti-mouse immunoglobulin horseradish-peroxidase secondary antibody was incubated for 30 mins. Visualisation used Fast-DAB (Sigma) according to manufacturer's instructions. Cells were counterstained with haemotoxylin.

Images were captured using a Nikon Eclipse E-1000 microscope attached to the Lucia image analysis system. Haemotoxylin stained nuclei were counted by the software using size-exclusion parameters which excluded small and large debris. Accuracy was determined within the software using binary overlays of the counted objects to examine if any non-nuclei counts occurred or if any nuclei were missed. It was found that the software was accurate to over 99%. DAB positive cells were counted by hand on the captured images. Counts are expressed as % positive cells/total cells.

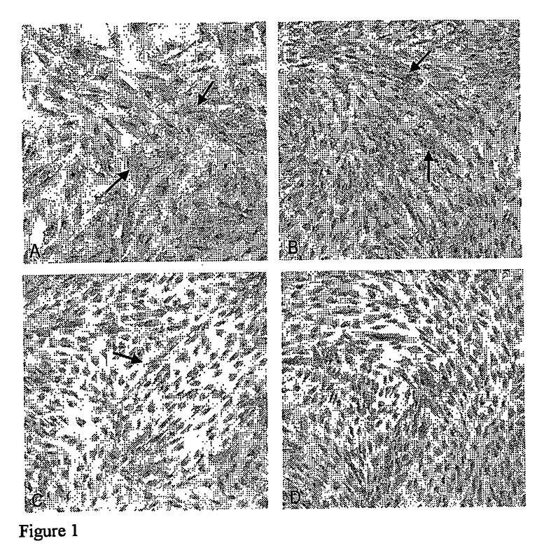

Experimental conditions were optimised over a number of experiments to produce myofibroblast numbers typically around 10% of the total cell number in control treatments. Myofibroblasts were easily identifiable compared to normal fibroblasts. Treatment with pioglitazone reduced the number of fibroblasts present at the end of the experimental period. This was apparent visually where control slides exhibited easily identifiable myofibroblasts compared to increasing doses of pioglitazone (FIG. 1).

The data presented in FIG. 1 indicate that pioglitazone reduced the number of myofibroblasts as a proportion of total cell number in a dose response fashion. Results are expressed as % myofibroblasts of total cells and ±S.E.M.

| Conc | % Myofibroblasts | SEM |
|---|---|---|
| 0 | 3.47 | 0.20276 |
| 0.1 | 1.81* | 0.11479 |
| 1 | 0.71** | 0.09152 |
| 10 | 0.10** | 0.04622 |

*p < 0.05
**p < 0.005.

### EXAMPLE 2

Morphology of Pioglitazone Treated Myofibroblasts In Vitro

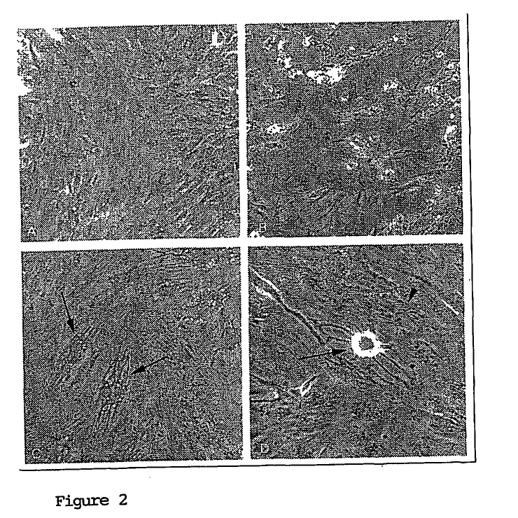

Morphology of fibroblasts and myofibroblasts after treatment with pioglitazone was assessed by phase contrast microscopy. See FIG. 2. The cell labelled with the arrow appears to be shrinking and therefore undergoing apoptosis. This cell appears to have a residual marked cytoskeleton when compared with the fibroblast marked with the arrowhead. The prominent cytoskeleton suggests that the cell labelled with the arrow is a myofibroblast (x200). These data suggest that there was abundant cell death at higher concentrations of pioglitazone and that the cells which were dying were myofibroblasts and this was mediated through apoptosis.

### EXAMPLE 3

Cell Shrinkage and Cytoplasmic Blebbing in Pioglitazone-Treated Myofibroblasts

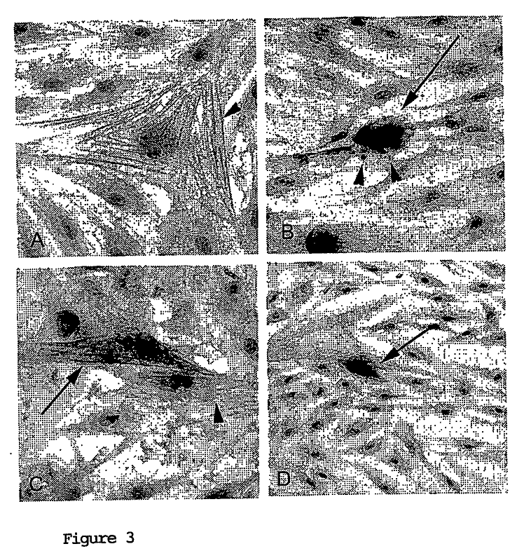

Human dermal fibroblasts were cultured and differentiated into myofibroblasts in myofibroblast differentiation medium supplemented with 1 ng/ml TGF-beta. In this experiment, differentiation took place in the presence of 1 uM pioglitazone. Results are shown in FIG. 3. As described in Example 1, pioglitazone reduced the number of myofibroblasts as a proportion of total cell number. Certain alpha-smooth muscle actin positive cells which were present did not show typical myofibroblast morphology and showed characteristics which were suggestive of apoptosis. This included membrane blebbing and cytoskeletal degradation.

1. Use of an activator of PPARγ in the manufacture of a medicament for the treatment of a condition affecting the skin characterised by disordered fibroblast or myofibroblast function, excessive matrix production, modular fasciitis or Dupuytren's Contracture.

2. Use according to claim 1, wherein the condition comprises keloids or hypertrophic scarring.

3. Use according to claim 1, wherein the condition is scleroderma.

4. Use according to claim 1, wherein the condition is nodular fasciitis or Dupuytren's Contracture.

5. Use according to any of claims 1 to 4, wherein the activator is a thiazolidinedione.

6. Use according to claim 5, wherein the activator is a 5-phenyl-thiazolidinedione.

7. Use according to claim 5, wherein the activator is a 5-benzyl-thiazolidinedione.

8. Use according to claim 5, wherein the activator is a 5-(naphthyl or benzothienyl)-thiazolidinedione.

9. Use according to any of claims 1 to 4, wherein the activator is pioglitazone, rosiglitazone, ciglitazone, troglitazone, isaglitazone, darglitazone or englitazone.

10. Use according to claim 9, wherein the activator is pioglitazone.

11. Use according to any of claims 1 to 4, wherein the activator is a 4-phenyloxyethyl-5-methyl-2-phenyloxazole.

12. Use according to any of claims 1 to 4, wherein the activator is 15-deoxy-delta-12, 14-prostaglandin J2.

13. Use according to any of claims 1 to 4, wherein the activator is a benzophenone derivative of structure I 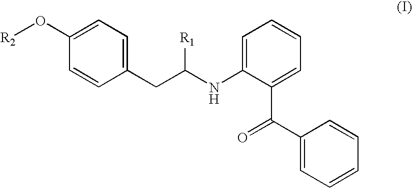
where R₁ is a carbocyclic acid or 5-membered heteroaryl group containing one or two nitrogen, oxygen or sulfur atoms and one or two nitrogen atoms, optionally substituted with a (C₁-C₃) alkyl group; and R₂ is 2-(5-methyl-2-phenyloxazol-4-yl)ethyl or 2-(2-pyridylmethylaminoethyl).

14. Use according to any of claims 1 to 4, wherein the activator is a sulphonamide derivative of structure II 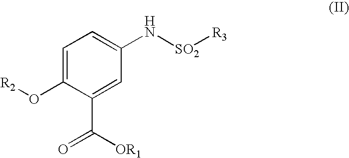
where R₁ is C₁-C₅ alkyl; R₂ is a halo-substituted pyridyl group, and R₃ is a phenyl group.

15. Use according to any of claims 1 to 14, wherein the medicament is adapted for topical administration.

16. Use according to any of claims 1 to 14, wherein the medicament is adapted for intralesional or subcutaneous administration.

17. Use according to any preceding claim, wherein the medicament comprises 1 to 400 mg of the activator.
